(19) United States
(12) Patent Application Publication
(10) Pub. No.: US 2021/0308092 A1
(43) Pub. Date: Oct. 7, 2021

(54) CANNABINOID COMPOSITION AND METHOD FOR TREATING PTSD AND/OR ANXIETY

(71) Applicant: Zelira Therapeutics Operations Pty Ltd, Perth, Western Australia (AU)

(72) Inventor: Harry KARELIS, Perth, Western Australia (AU)

(21) Appl. No.: 17/257,768

(22) PCT Filed: Jul. 3, 2019
(86) PCT No.: PCT/AU2019/050698

§ 371 Date: Jan. 4, 2021

(30) Foreign Application Priority Data
Jul. 3, 2018 (AU) 2018100925

(51) Int. Cl. A61K 31/352; A61K 31/05; A61K 31/015; A61K 31/045; A61K 31/01; A61K 36/185; A61K 9/00; A61K 9/48; A61P 25/22

(52) U.S. Cl. CPC A61K 31/352; A61K 31/05; A61K 31/015; A61K 31/045; A61P 25/22; A61K 36/185; A61K 9/0053; A61K 9/006; A61K 9/4808; A61K 31/01

(57) ABSTRACT

The invention relates to pharmaceutical compositions comprising Δ⁹-tetrahydrocannabinol (THC), cannabidiol (CBD) and a terpene fraction obtained by extraction of a Cannabis plant, and their use in the treatment of anxiety and/or post-traumatic stress disorder (PTSD).

### FIELD

The invention relates to pharmaceutical compositions comprising cannabidiol (CBD) and Δ⁹-tetrahydrocannabinol (THC), and their use to treat post-traumatic stress disorder (PTSD) and/or anxiety.

### BACKGROUND

The biological activity of Cannabis is well known and has led it to become a “recreational” drug. However, with the discovery of a class of cannabinoid (CB) receptors, and the relaxation of laws regulating Cannabis use—in some jurisdictions decriminalisation—there now exists the opportunity to explore the potential of Cannabis as a source of new therapeutics.

The two main receptors implicated in the endocannabinoid system are cannabinoid receptors 1 and 2 (CB1 and CB2, respectively). Both CB1 and CB2 belong to the G-protein coupled receptor (GPCR) class of transmembrane receptors.

THC is the main psychotropic constituent of Cannabis, its main pharmacological effects including analgesia, muscle relaxation, antiemesis, appetite stimulation and psychoactivity. THC mimics the action of the endogenous cannabinoid receptor ligands. THC is a partial agonist of CB1 receptors, which are primarily expressed in the central nervous system, including within the brain. Further, due to its well-documented psychotropic efficacy, it is known that THC can cross the blood brain barrier when administered in a variety of forms, including inhalation and oral dosing.

CBD is the main non-psychotropic cannabinoid present in the Cannabis sativa plant, in some cases constituting up to 40 per cent of its extract depending on extraction technique. Both animal and human studies suggest that the pharmacokinetics and pharmacodynamics of CBD are very complex. CBD appears to operate at both CB1 and CB2 endocannabinoid receptors within the endocannabinoid system (ECS) indirectly stimulating endogenous cannabinoid signalling (anadamide) by suppressing fatty acid amide hydrolase (FAAH), the enzyme that breaks down anandamide. Importantly, this enables more anandamide to remain at the receptors, which elicits anxiolytic and antidepressant like effects. This indirect agonist property at the cannabinoid receptors may also explain its promising safety profile. Furthermore, CBD has been shown to also act on the vanilloid, adenosine and serotonin receptors explaining its broad spectrum of potential therapeutic properties in animal models and humans, including anxiolytic, antidepressant, neuroprotective, anti-inflammatory and immunomodulatory actions.

There is evidence that THC and CBD used in combination, act synergistically in the treatment of some indications, such as to maximize an analgesic response. CBD has been demonstrated to antagonise some undesirable effects of THC including intoxication, sedation and tachycardia, while contributing analgesic, anti-emetic, and anti-carcinogenic properties.

While there is growing preclinical data showing the plausibility of the use of cannabinoids in treating post-traumatic stress disorder (PTSD), the research is piecemeal and remains limited. However, randomised controlled trials using THC, CBD and THC+CBD are underway (NCT02759185 and NCT02517424) and the animal studies have provided neurobiological and experimental data that has suggested that CBD and THC+CBD may modulate fear memory in PTSD and may have acute positive effects on anxiety (Loflin et al., Current Opinion in Psychology, 2017, 14:78-83).

Accordingly, there is a continuing need to provide alternative pharmaceutical compositions comprising THC and CBD, including those that may be used in the treatment of mental disorders, such as post-traumatic stress disorder and anxiety.

### SUMMARY

The inventors believe that patients suffering from post-traumatic stress disorder and/or anxiety may be treated with a pharmaceutical composition comprising Δ⁹-tetrahydrocannabinol (THC) and cannabidiol (CBD) and a terpene fraction obtained by extraction of a Cannabis plant.

In one aspect, there is provided a pharmaceutical composition comprising THC, CBD and a terpene fraction obtained by extraction of a Cannabis plant.

In another aspect, there is provided a method of treating PTSD and/or anxiety, comprising administering to a subject in need thereof an effective amount of the pharmaceutical composition of the invention. In some embodiments, the method comprises administering an effective amount of a Cannabis extract comprising THC as primary cannabinoid and a terpene fraction and administering a Cannabis extract comprising CBD as primary cannabinoid and a terpene fraction.

In a further aspect, there is provided a kit of parts comprising in separate parts (a) a pharmaceutical composition comprising THC and a terpene fraction, and (b) a pharmaceutical composition comprising CBD and a terpene fraction.

In yet another aspect, there is provided use of one or more of (a) a Cannabis extract, (b) THC and (c) CBD in the manufacture of a medicament for treating PTSD and/or anxiety, wherein the medicament comprises THC, CBD and a terpene fraction.

### DEFINITIONS

The term “cannabinoid” as used herein relates to any compound that has been isolated from a Cannabis plant or synthetically created that has activity involving the endocannabinoid system. The term is used to describe the relevant compound itself irrespective of its source.

The term “cannabinoid fraction” is used to describe the combination of cannabinoid compounds present in the Cannabis extract.

The term “terpenes” or “terpenoids” as used herein refers to a class of hydrocarbon molecules, which often provide a unique smell. Terpenes are derived from units of isoprene, which has the molecular formula C₅H₈. The basic molecular formula of terpenes are multiples of the isoprene unit, i.e. (C₅H₈)ₙ, where n is the number of linked isoprene units. Terpenoids are terpene compounds that have been further metabolised in the plant, typically through an oxidative process, and therefore usually contain at least one oxygen atom.

The term “terpene fraction” is used to describe the combination of terpene and terpenoid compounds present in the Cannabis extract.

As used herein, the terms “treating”, “treatment”, “treat” and the like mean affecting a subject, patient, tissue or cell to obtain a desired pharmacological and/or physiological effect. The effect may be prophylactic in terms of completely or partially preventing, or reducing the severity of, a disease or associated symptom, and/or may be therapeutic in terms of a partial or complete cure of a disease.

The term “administering” refers to providing the pharmaceutical composition to a patient suffering from or at risk of the disease(s) or condition(s) to be treated or prevented.

By “effective amount” it is meant an amount sufficient that, when administered to the patient, an amount of the drug is provided to achieve an effect. In the case of a therapeutic method, this effect may be the treatment of the specified disease and/or condition or a symptom thereof. Therefore, the “effective amount” may be a “therapeutically effective amount”. By “therapeutically effective amount” it is meant an amount sufficient that when administered to the patient an amount of active ingredient is provided to treat the disease or a symptom of the disease.

As used herein and in the appended claims, the singular forms “a,” “an,” and “the” include plural reference unless the context clearly dictates otherwise. Thus, for example, a reference to “an excipient” may include a plurality of excipients, and a reference to “a subject” may be a reference to one or more subjects, and so forth.

The term “(s)” following a noun contemplates the singular or plural form, or both.

The term “and/or” can mean “and” or “or”.

Unless the context requires otherwise, all percentages referred to herein are percentages by weight of the composition.

Various features of the invention are described and/or claimed with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, preferably means an amount within ±25%, ±10%, ±5%, ±1% or ±0.1% of that value.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

Before describing the present invention in detail, it is to be understood that this invention is not limited to particularly exemplified pharmaceutical compositions, methods of production or treatment, which may, of course, vary. It is also to be understood that the terminology used herein is for the purpose of describing particular embodiments of the invention only, and is not intended to be limiting.

The inventions described and claimed herein have many attributes and embodiments including, but not limited to, those set forth or described or referenced in this summary section, which is not intended to be all-inclusive. The inventions described and claimed herein are not limited to or by the features or embodiments identified in this summary section, which is included for purposes of overview illustration only and not limitation.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

### DESCRIPTION OF EMBODIMENT(S)

The present invention provides a pharmaceutical composition comprising THC and CBD and a terpene fraction obtained from extraction of a Cannabis plant. The invention also provides pharmaceutical compositions comprising THC and the terpene fraction (including those substantially free of CBD), and pharmaceutical compositions comprising CBD and the terpene fraction (including those substantially free of THC).

It is believed that the synergistic effects of co-administration of THC and CBD together may be further enhanced (e.g. synergistically) by the co-administration of a terpene fraction obtained by extraction of a Cannabis plant.

The pharmaceutical compositions comprising both THC and CBD may comprise THC and CBD in ratio of THC:CBD from about 10:1 to about 1:10. In some embodiments, the ratio of THC:CBD will be balanced, for example from about 2:1 to about 1:2, such as about 0.8:1 to about 1.2:1 or about 1:1.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The ratio of THC to CBD may be readily determined by methods known in the art, including High-Performance Liquid Chromatography (HPLC) and Ultra Performance Liquid Chromatography (UPLC).

In some embodiments, pharmaceutical compositions comprising THC may comprise THC in a minimum amount of at least about 15 wt %, for example, at least about 25 wt %, about 35 wt % or about 40 wt %. In some embodiments, the pharmaceutical composition comprises THC in a maximum amount of up to about 85 wt %, about 80 wt %, about 75 wt %, about 70 wt %, about 65 wt %, about 60 wt %, about 55 wt %, about 50 wt %, about 45 wt %, about 40 wt %, about 35 wt %, about 30 wt %, about 25 wt % or about 20 wt %. It will be appreciated that the amount of THC may be within the range from any of these minimum amounts to any of these maximum amounts. All combinations of these minimum and maximum amounts are contemplated. For example, in some embodiments, the pharmaceutical composition comprises THC in an amount of from about 15 wt % to about 85 wt %, about 15 wt % to about 75 wt % or about 40 wt % to about 60 wt %.

In some embodiments, pharmaceutical compositions comprising CBD may comprise CBD in a minimum amount of at least about 15 wt %, for example, at least about 25 wt %, about 35 wt % or about 40 wt %. In some embodiments, the pharmaceutical composition comprises CBD in a maximum amount of up to about 60 wt %, about 55 wt %, about 50 wt %, about 45 wt %, about 40 wt %, about 35 wt %, about 30 wt %, about 25 wt % or about 20 wt %. It will be appreciated that the amount of CBD may be within the range from any of these minimum amounts to any of these maximum amounts. All combinations of these minimum and maximum amounts are contemplated. For example, in some embodiments, the pharmaceutical composition comprises CBD in an amount of from about 15 wt % to about 60 wt %, about 15wt % to about 55 wt % or about 40 wt % to about 55 wt %.

In some embodiments, the pharmaceutical composition comprises THC and CBD in a minimum total amount of at least about 30 wt %, for example, at least about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt % or about 99 wt %. In some embodiments, the pharmaceutical composition comprises THC and CBD in a total maximum amount of up to about 99 wt %, for example, up to about 95 wt %, about 90 wt %, about 85 wt %, about 80 wt %, about 70 wt %, about 60 wt %, about 50 wt %, about 40 wt %, about 30 wt %, about 20 wt % or about 15wt %. It will be appreciated that the total amount of CBD and THC may be within the range from any of these minimum amounts to any of these maximum amounts. All combinations of these minimum and maximum amounts are contemplated. For example, in some embodiments, the pharmaceutical composition comprises CBD and THC in an amount of from about 30 wt % to about 99 wt %.

References to THC and CBD (and any other natural product, including cannabinoid(s), terpene(s) and terpenoid(s)) used herein include the relevant compound and pharmaceutically acceptable salts and/or solvates (including hydrates) thereof.

The THC and CBD may be combined from purified forms of the compounds, which may be purified after extraction from a natural source or produced synthetically or semi-synthetically. Any means known in the art for producing purified forms of CBD and/or THC is contemplated.

In some embodiments, the terpene fraction and at least a portion of the THC and CBD of the pharmaceutical composition are provided in the form of a Cannabis extract. To this extract may be added purified THC or CBD to achieve the desired cannabinoid ratio, or two or more Cannabis extracts may be combined to achieve the desired cannabinoid ratio. In some embodiments, the pharmaceutical composition comprises a mixture of two or more Cannabis extracts comprising THC, CBD and a terpene fraction. Typically, the two or more extracts provide different cannabinoid ratios. The combined Cannabis extracts may therefore be obtained from different Cannabis varieties, from different parts of the same Cannabis variety or obtained using different extraction conditions/techniques.

Cannabis plants produce a diverse array of secondary metabolites, including cannabinoids, terpenes, terpenoids, sterols, triglycerides, alkanes, squalenes, tocopherols, carotenoids and alkaloids. The mix of these secondary metabolites varies depending on several factors, including Cannabis variety, part of the Cannabis plant extracted, method of extraction, processing of the extract and season.

There are several varieties of Cannabis plant, which have been described under two distinct naming conventions. One of these conventions identifies three distinct species of Cannabis plant, namely Cannabis sativa Linnaeus, Cannabis indica LAM., and Cannabis ruderalis. Another convention identifies all Cannabis plants as belonging to the Cannabis sativa L. species, with the various varieties divided amongst several subspecies, including: Cannabis sativa ssp. sativa and ssp. indica. As used herein, the term “Cannabis” refers to any and all of these plant varieties.

Extracts of Cannabis may be prepared by any means known in the art. The extracts may be formed from any part of the Cannabis plant containing cannabinoid and terpene and/or terpenoid compounds. Extracts may be formed from a leaf, seed, trichome, flower, keif, shake, bud, stem or a combination thereof. The part of the Cannabis plant may be used fresh or dried prior to extraction. All known means of drying the plant material are contemplated. In some embodiments, the extract is formed by contacting any part of the Cannabis plant with an extractant. Any suitable extractant known in the art may be used, including, for example, alcohols (e.g. methanol, ethanol, propanol, butanol, propylene glycol etc.), water, hydrocarbons (e.g. butane, hexane, etc.), oils (e.g. olive oil, vegetable oil, essential oil, etc.), a polar organic solvent (e.g. ethyl acetate, polyethylene glycol, etc.) or a supercritical fluid (e.g. liquid CO₂). The extractant may be completely or partially removed prior to incorporation of the Cannabis extract into the pharmaceutical composition, or it may be included in the pharmaceutical composition as a carrier. The extractant may be removed by heating the extract optionally under reduced pressure (e.g. under vacuum). It will be appreciated that some of the more volatile plant metabolites (such as terpenes) may also be removed with the extractant. Accordingly, in some embodiments, removing the extractant may enrich the cannabinoid fraction of the extract. In some embodiments, the extract is filtered to remove particulate material, for example, by passing the extract through filter paper or a fine sieve (e.g. a sieve with pore sizes of 5 μm).

In some embodiments, the Cannabis extract is formed by applying heat and/or pressure to the plant material. Typically, in these embodiments, no extractant is required.

In some embodiments, one or more additional compounds (e.g. cannabinoid, terpene or terpenoid compounds) may be added to the Cannabis extract. The addition of compounds may be to compensate for natural variations in the relative amounts of certain compounds being expressed in the Cannabis plant. The added compounds may be synthetic versions of the desired compounds, they may be purified compounds obtained from other Cannabis extracts, or they may be added by blending two or more Cannabis extracts.

The cannabinoid fraction typically accounts for the majority of the compounds present in the Cannabis extract.

In some embodiments, the Cannabis extract may comprise about 35% to about 95% by weight cannabinoids, for example, about 40% to about 90%, about 45% to about 70% or about 45% to about 55% by weight of the Cannabis extract. In some embodiments, the Cannabis extract comprises about 5% to about 65% by weight of non-cannabinoids, for example, about 5% to about 50%, about 10% to about 40% by weight or about 15% to about 30% by weight non-cannabinoids.

The cannabinoid fraction of a Cannabis extract may comprise a primary (or main) cannabinoid. As used herein, the term “primary cannabinoid” relates to the cannabinoid present in a Cannabis extract is the highest concentration. Typically, the primary cannabinoid may be Δ⁹-tetrahydrocannabinol (THC) or cannabidiol (CBD). The primary cannabinoid may be present in the Cannabis extract in an amount of at least about 0.1%, about 0.5%, about 1%, about 1.5%, about 2%, about 2.5%, about 3%, about 3.5%, about 4%, about 20%, about 25%, about 30%, about 35%, about 40%, about 45%, about 50% or about 55% by weight of the Cannabis extract. Accordingly, when THC is the primary cannabinoid, the Cannabis extract may comprise at least about 0.1%, about 0.5%, about 1%, about 1.5%, about 2%, about 2.5%, about 3%, about 3.5%, about 4%, about 4.5%, about 20%, about 25%, about 30%, about 35%, about 40%, about 45%, about 50% or about 55% by weight Δ⁹-tetrahydrocannabinol (THC), for example, about 0.1% to about 97%, about 0.1% to about 20%, or about 50 to about 90% by weight of Δ⁹-tetrahydrocannabinol (THC). When CBD is the primary cannabinoid, the Cannabis extract may comprise at least about 0.1%, about 0.5%, about 1%, about 1.5%, about 2%, about 2.5%, about 3%, about 3.5%, about 4%, about 20%, about 25%, about 30%, about 35%, about 40%, about 45%, about 50%, about 55% or about 60% by weight CBD, for example, about 0.1% to about 97%, about 0.1% to about 10% or about 50 to about 90% by weight CBD.

In addition to the primary cannabinoid, the Cannabis extract may further comprise a secondary cannabinoid. As used herein, the term “secondary cannabinoid” relates to the cannabinoid present in a Cannabis extract is the second highest concentration. The secondary cannabinoid is therefore present in the Cannabis extract in an amount less than the primary cannabinoid. In some embodiments where the primary cannabinoid is THC, the secondary cannabinoid may be CBD. In some embodiments where the primary cannabinoid is CBD, the secondary cannabinoid may be THC. The secondary cannabinoid may be present in the Cannabis extract in an amount of at least about 0.001% by weight, for example, at least about 0.005%, 0.01%, 0.05%, 0.1%, 0.5%, 1%, 1.5% or 2% by weight of the extract. The secondary cannabinoid may be present in a maximum amount of less than the amount of the primary cannabinoid, such as up to about 10%, for example, up to about 9%, 8%, 7%, 6%, 5% by weight of the extract. It will be appreciated that the amount of secondary cannabinoid may be within the range from any of these minimum amounts to any of these maximum amounts

In some embodiments, the Cannabis extract is enriched in one or the other of CBD or THC. It has been shown that endocannabinoids (i.e. naturally occurring cannabinoids), including CBD and THC, interact with a class of G protein-coupled receptors (GPCRs) named the “cannabinoid receptors”, e.g. the CB1 or CB2 receptors. However, structurally related cannabinoid compounds may have vastly different activity. Despite these differences in activity, the present invention relies on the activity of the combination of THC, CBD and the terpene fraction.

In some embodiments, the Cannabis extract may comprise at least about 0.001% by weight THC and/or CBD, for example, from about 0.001% to about 99.999% by weight THC and/or CBD, at least about 0.001% to about 20% by weight THC and/or CBD, about 0.01% to about 20% by weight THC and/or CBD, about 0.01% to about 15% by weight THC and/or CBD.

In some embodiments, the Cannabis extract may comprise THC and CBD in a combined weight of at least about 1% by weight, for example, at least about 5% by weight. In some embodiments, the combined amount of CBD and THC may be 1-20%, 1-15%, 6-11% or 50-90% by weight of the pharmaceutical composition. The ratio of THC to CBD may be from about 100:0 to about 0:100, about 100:1 to about 1:100, about 80:1 to about 1:80, about 60:1 to about 1:60, about 40:1 to about 1:40 or about 20:1 to about 1:20. In some embodiments, the ratio of THC to CBD may be balanced, for example in a ratio of THC:CBD of about 2:1 to about 1:2 or about 1:1. The ratio of THC:CBD may be expressed as a single number by dividing the amount of THC by the amount of CBD present. Accordingly, the ratio of THC:CBD in the pharmaceutical compositions may be 0.001, 0.1, 0.2, 0.3, 0.4, 0.45, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or higher. In some embodiments, the ratio of THC:CBD in a Cannabis extract may be between any of these values, for example, from 0.001 to 3, 0.2 to 3 or 0.4 to 2.6.

Embodiments of the pharmaceutical composition comprising a balanced amount of THC and CBD may be obtained by, for example, adding CBD to a Cannabis extract that comprises THC as primary cannabinoid, adding THC to a Cannabis extract that comprises CBD as primary cannabinoid, or combining a Cannabis extract comprising THC as primary cannabinoid with a Cannabis extract comprising CBD as primary cannabinoid.

Embodiments of the pharmaceutical composition enriched in one or the other of THC or CBD may be obtained by, for example, adding purified or synthetic THC or CBD to a Cannabis extract, to obtain the desired amount of THC or CBD or the desired ratio of THC to CBD.

Typically, the Cannabis extract may also comprise other cannabinoids in addition to THC and/or CBD, such as any of the cannabinoids previously identified in Cannabis plants. To date, over 100 cannabinoids have been identified in Cannabis plants. A comprehensive list of these cannabinoids may be found in Mahmoud A. El Sohly and Waseem Gul, “Constituents of Cannabis Sativa.” In Handbook of Cannabis Roger Pertwee (Ed.) Oxford University Press (2014) (ISBN: 9780199662685). Cannabinoids that have been identified in Cannabis plants include: Cannabigerol (E)-CBG-C5, Cannabigerol monomethyl ether (E)-CBGM-C5 A, Cannabigerolic acid A (Z)-CBGA-C5 A, Cannabigerovarin (E)-CBGV-C3, Cannabigerolic acid A (E)-CBGA-C5 A, Cannabigerolic acid A monomethyl ether (E)CBGAM-C5 A and Cannabigerovarinic acid A (E)-CBGVAC3A; (±)-Cannabichromene CBC-C5, (±)-Cannabichromenic acid A CBCA-C5 A, (±)-Cannabivarichromene, (±)-Cannabichromevarin CBCV-C3, (±)-Cannabichromevarinic acid A CBCVA-C3 A; (−)-Cannabidiol CBD-C5, Cannabidiol momomethyl ether CBDMCS, Cannabidiol-C4 CBD-C4, (−)-Cannabidivarin CBDVC3, Cannabidiorcol CBD-CI, Cannabidiolic acid CBDA-C5, Cannabidivarinic acid CBDVA-C3; Cannabinodiol CBNDCS, Cannabinodivarin CBND-C3; Δ⁹-Tetrahydrocannabinol Δ⁹-THC-C5, Δ⁹-Tetrahydrocannabinol-C4 Δ⁹-THCC4, Δ⁹-Tetrahydrocannabivarin Δ⁹-THCV-C3, Δ⁹-Tetrahydrocannabiorcol Δ⁹-THCO-CI, Δ⁹-Tetrahydrocannabinolic acid A Δ⁹-THCA-C5 A, Δ⁹-Tetrahydrocannabinolic acid B Δ⁹-THCA-C5 B, Δ⁹-Tetrahydrocannabinolic acid-C4 A and/or B Δ⁹-THCA-C4 A and/or B, Δ⁹-Tetrahydro-cannabivarinic acid A Δ⁹-THCVA-C3 A, Δ⁹-Tetrahydrocannabiorcolic acid A and/or B Δ⁹-THCOA-CI A and/or B), (−)-Δ⁸-trans-(6aR,10aR)-Δ⁸-Tetrahydrocannabinol Δ⁸-THC-C5, (−)-Δ⁸-trans-(6aR,10aR)-Tetrahydrocannabinolic acid A Δ⁸-THCA-C5 A, (−)-(6aS,10aR)-Δ⁹-Tetrahydrocannabinol (−)-cis-Δ⁹-THC-C5; Cannabinol CBN-C5, Cannabinol-C4 CBN-C4, Cannabivarin CBN-C3, Cannabinol C2 CBN-C2, Cannabiorcol CBN-CI, Cannabinolic acid A CBNA-C5 A, Cannabinol methyl ether CBNM-C5, (−)-(9R,10R)-trans-Cannabitriol (−)-trans-CBT-C5, (+)-(9S,10S)-Cannabitriol (+)-trans-CBT-C5, (±)-(9R,10S/9S,10R)−); Cannabitriol (±)-cis-CBT-C5, (−)-(9R,10R)-trans-10-O-Ethyl-cannabitriol (−)-trans-CBT-OEt-C5, (±)-(9R,10R/9S,10S)-Cannabitriol-C3 (±)-trans-CBT-C3, 8,9-Dihydroxy-Δ⁶a(10a)-tetrahydrocannabinol 8,9-Di-OH-CBT-C5, Cannabidiolic acid A cannabitriol ester CBDA-C5 9-OH-CBT-C5 ester, (−)-(6aR,9S,10S,10aR)-9,10-Dihydroxyhexahydrocannabinol, Cannabiripsol, Cannabiripsol-C5, (−)-6a,7,10a-Trihydroxy-Δ⁹-tetrahydrocannabinol (−)-Cannabitetrol, 10-Oxo-Δ^{6a}(10a)tetrahydrocannabinol OTHC); (5aS,6S,9R,9aR)-Cannabielsoin CBE-C5, (5aS,6S,9R,9aR)-C3-Cannabielsoin CBE-C3, (5aS,6S,9R,9aR)-Cannabielsoic acid A CBEA-C5 A, (5aS,6S,9R,9aR)-Cannabielsoic acid B CBEA-C5 B; (5aS,6S,9R,9aR)-C3-Cannabielsoic acid B CBEA-C3 B, Cannabiglendol-C3 OH-iso-HHCV-C3, Dehydrocannabifuran DCBF-C5, Cannabifuran CBF-C5), (−)-Δ⁷-trans-(1R,3R,6R)-Isotetrahydrocannabinol, (±)-Δ⁷-1,2-cis-(1R,3R,6S/1S,3S,6R)-Isotetrahydrocannabivarin, (−)-Δ⁷-trans-(1R,3R,6R)-Isotetrahydrocannabivarin; (±)-(IaS,3aR,8bR,8cR)-Cannabicyclol CBL-C5, (±)-(IaS,3aR,8bR,8cR)-Cannabicyclolic acid A CBLA-C5 A, (±)-(IaS,3aR,8bR,8cR)-Cannabicyclovarin CBLV-C3; Cannabicitran CBTCS; Cannabichromanone CBCN-C5, CannabichromanoneC3 CBCN-C3, and Cannabicoumaronone CBCON-C5.

In some embodiments, the Cannabis extract further comprises one or more of Δ⁹-Tetrahydrocannabinolic acid (THCA), Δ⁹-Tetrahydrocannabivarin (THCV), (−)-Cannabidivarin (CBDV), Cannabinodiol (CBN) and Cannabigerol (CBG). Each of these cannabinoids may be present in an amount from about 0.001% to about 40% by weight of the Cannabis extract. Typically, the other cannabinoids are present in amounts lower than the primary cannabinoid or, if present, the secondary cannabinoid(s).

In some embodiments, certain cannabinoids may be absent, or present in non-detectable amounts (e.g. less than about 0.001% by weight of the analyte). In some embodiments, the Cannabis extract may exclude one or more of the following cannabinoids: Δ⁹-Tetrahydrocannabinolic acid (THCA), Δ⁹-Tetrahydrocannabivann (THCV), Cannabidiolic acid (CBDA), Cannabinodiol (CBN), (−)-Cannabidivarin (CBDV), Cannabigerol (CBG) and Cannabichromene (CBC).

Cannabis extracts may further comprise a non-cannabinoid fraction. The non-cannabinoid fraction may include a terpene fraction. In some embodiments, the Cannabis extract comprises a terpene fraction in an amount of less than about 50% by weight, for example, less than about 45%, about 40%, about 35%, about 30%, about 25%, about 20%, about 15%, about 10%, about 9%, about 8%, about 7%, about 6%, about 5%, about 4%, about 3%, about 2% or about 1% by weight of the extract. In some embodiments, the Cannabis extract may comprise terpene and terpenoid compounds in an amount of at least about 0.001% by weight of the extract, for example, at least about 0.005%, about 0.01%, about 0.05%, about 0.1%, about 0.2%, about 0.25%, about 0.3%, about 0.35%, about 0.4%, about 0.45%, about 0.5%, about 1%, about 2%, about 3%, about 4%, about 5%, about 6%, about 10%, about 15% or more of the total weight of the extract. In some embodiments, the the pharmaceutical composition comprises about 0.001% to about 50% by weight of terpene and terpenoid compounds, for example, about 0.01% to about 50% by weight, about 0.01% to about 10% by weight, about 0.01% to about 6% by weight or about 0.01 to about 5% by weight of the pharmaceutical composition.

Typically, the terpene fraction in the plant material used to form the extract may have a different terpene/terpenoid profile than the terpene profile of the final extract, both in terms of the amounts of specific compounds in the terpene fraction and the weight of the terpene fraction relative to the other components. For example, a Cannabis flower may comprise about 20% by weight cannabinoids and about 3% by weight terpenes. Following extraction and concentration (i.e. removal of the extractant), the cannabinoid fraction may amount to about 50-90% by weight and the terpene fraction may amount to about 0.1-6% by weight of the Cannabis extract. This typical scenario shows that the cannabinoids are concentrated when the extractant is removed, the relative amount of the terpene fraction is reduced, likely due to the volatility of many of the terpenes/terpenoids present in the terpene fraction. Therefore, the profile of the terpene fraction present in the Cannabis extract is significantly different from the profile of the terpene fraction that exists in Nature.

The efficacy of a composition may be enhanced when the terpene fraction has a certain profile, i.e. a certain proportion of particular terpenes/terpenoids are present in the extract. It is believed that the increase in efficacy may be synergistic (i.e. non-additive). It is also believed that the presence of specific components in the terpene fraction may enhance the patient's tolerance to cannabinoid therapy.

A variety of terpenes and terpenoids have also been identified in Cannabis extracts, including monoterpenes, monoterpenoids, sesquiterpenes and sesquiterpenoids. For example, the following terpenes and terpenoids have been identified in Cannabis extracts: Alloaromadendrene, allyl hexanoate, benzaldehyde, (Z)-a-cis-bergamotene, (Z)-a-trans-bergamotene, β-bisabolol, epi-α-bisabolol, ↑-bisabolene, borneol (camphol), cis-γ-bisabolene, bomeol acetate (bomyl acetate), α-cadinene, camphene, camphor, cis-carveol, caryophyllene (β-caryophyllene), α-humulene (α-caryophyllene), γ-cadinene, Δ-3-carene, caryophyllene oxide, 1,8-cineole, citral A, citral B, cinnameldehyde, α-copaene (aglaiene), γ-curcumene, β-cymene, p-cymene, β-elemene, γ-elemene, ethyl decadienoate, ethyl maltol, ethyl propionate, ethylvanillin, eucalyptol, α-eudesmol, β-eudesmol, γ-eudesmol, eugenol, cis-β-farnesene ((Z)-β-farnesene), trans-α-farnesene, trans-β-farnesene, trans-γ-bisabolene, fenchone, fenchol (norbomanol, β-fenchol), geraniol, α-guaiene, guaiol, gurjunene, methyl anthranilate, methyl salicylate, 2-methyl-4-heptanone, 3-methyl-4-heptanone, hexyl acetate, ipsdienol, isoamyl acetate, lemenol, limonene, d-limonene (limonene), linolool (linalyl alcohol, β-linolool), α-longipinene, menthol, γ-muurolene, myrcene (β-myrcene), nerolidol, trans-nerolidol, nerol, β-ocimene (cis-ocimene), octyl acetate, α-phellandrene, phytol, α-pinene (2-pinene), β-pinene, pulegone, sabinene, cis-sabinene hydrate (cis-thujanol), β-selinene, α-selinene, γ-terpinene, terpinolene (isoterpine), terpineol (α-terpineol), terpineol-4-ol, α-terpinene (terpilene), α-thujene (origanene), vanillin, viridiflorene (ledene), and α-ylange. In some embodiments, the pharmaceutical composition comprises one or more of these terpenes and/or terpenoids.

In some embodiments, the Cannabis extract may comprise one or more of: d-limonene, α-pinene, β-caryophyllene, linalool, β-myrcene, p-cymene, gurjunene, a nerolidol (e.g. nerolidol 1 and/or 2), α-terpinene, α-phellandrene, camphene, terpinolene, 1,8-cineole, γ-terpinene, guaiol, β-ocimene, β-pinene, γ-cadinene, caryophyllene oxide and β-farnesene. For example, the Cannabis extract may comprise one, two, three, four, five or more of these terpenes/terpenoids. Each of these terpenoids may be absent or may be present in an amount in the range of 0.001% to 50 % by weight of the terpene fraction.

In some embodiments, the terpene fraction comprises at least one of d-limonene, α-pinene, β-caryophyllene, linalool, β-myrcene, p-cymene, gurjunene and a nerolidol, especially at least two, at least three or at least four of these terpene/terpenoids.

In some embodiments, the terpene fraction comprises at least one of d-limonene, α-pinene, β-caryophyllene and linalool, especially at least two, at least three or at least four of these terpene/terpenoids.

In some embodiments, the terpene fraction may comprise one of the following combinations: d-limonene and α-pinene; d-limonene and β-caryophyllene; d-limonene and linalool; α-pinene and β-caryophyllene; α-pinene and linalool; β-caryophyllene and linalool, d-limonene, α-pinene and β-caryophyllene; d-limonene, α-pinene and linalool; d-limonene, β-caryophyllene and linalool; α-pinene, β-caryophyllene and linalool; and d-limonene, α-pinene, β-caryophyllene and linalool.

In some embodiments, specific terpenes or terpenoids may be absent, or present in non-detectable amounts (e.g. less than about 0.001% by weight of the analyte).

The identity and amounts of terpenes and/or terpenoids obtained by extraction of a Cannabis plant may be determined by methods known in the art, including gas chromatography (GC). Typically, the profile of a cannabinoid fraction and a terpene fraction of a Cannabis extract are determined separately using different analytical techniques.

The pharmaceutical composition comprises THC, CBD and a terpene fraction. In some embodiments, the pharmaceutical composition consists of a Cannabis extract and optionally one or more pharmaceutically acceptable excipients, such as a carrier. In some embodiments, the pharmaceutical composition comprises a Cannabis extract to which has been added one or more of THC, CBD, terpenes and/or terpenoids. The addition of compounds may be to compensate for natural variations in the relative amounts of certain compounds being expressed in the Cannabis plant or may be to enhance the activity of one or more cannabinoid, terpene or terpenoid compounds present in the extract or to provide the desired amount of the compound that is added. Terpenes and/or terpenoids may be added to adjust their content in the pharmaceutical composition to compensate for loss during an extraction process or to provide a desired non-natural terpene/terpenoid content in the pharmaceutical composition. The added compounds may be synthetic versions of the desired compounds, they may be purified compounds obtained from other Cannabis extracts or from other plant extracts, or they may be added by blending two or more Cannabis extracts.

In some embodiments, the pharmaceutical composition optionally comprises one or more pharmaceutically acceptable excipient(s). The excipient may be a carrier, diluent, adjuvant, or other excipient, or any combination thereof, and “pharmaceutically acceptable” meaning that they are compatible with the other ingredients of the pharmaceutical composition and are not deleterious to a patient upon or following administration. The pharmaceutical compositions may be formulated, for example, by employing conventional solid or liquid vehicles or diluents, as well as pharmaceutical additives of a type appropriate to the mode of desired administration (for example, excipients, binders, preservatives, stabilisers, flavours, etc.) according to techniques such as those well known in the art of pharmaceutical formulation (See, for example, Remington: The Science and Practice of Pharmacy, 21st Ed., 2005, Lippincott Williams & Wilkins). The pharmaceutically acceptable carrier may be any carrier included in the United States Pharmacopeia/National Formulary (USP/NF), the British Pharmacopoeia (BP), the European Pharmacopoeia (EP), or the Japanese Pharmacopoeia (JP). In some embodiments, the excipient may be non-natural (e.g. synthetically produced).

The pharmaceutical composition includes those suitable for oral, rectal, nasal, topical (including oro-mucosal such as buccal and sublingual), vaginal or parenteral (including intramuscular, sub-cutaneous and intravenous) administration or in a form suitable for administration by inhalation or insufflation.

The ingredients of the pharmaceutical composition may be placed into the form of pharmaceutical compositions and unit dosages thereof, and in such form may be employed as solids, such as tablets or filled capsules or syringes, or liquids such as solutions, suspensions, emulsions, elixirs, or capsules filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active ingredient(s), and such unit dosage forms may contain any suitable effective amount of the active ingredients commensurate with the intended daily dosage range to be employed.

For preparing pharmaceutical compositions described herein, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Tablets, powders, capsules, pills, cachets, and lozenges can be used as solid forms suitable for oral administration.

Liquid form preparations include solutions, dispersions, suspensions, and emulsions, for example, water or water-propylene glycol solutions or oils such as vegetable oils. For example, parenteral injection liquid preparations can be formulated as solutions in aqueous polyethylene glycol solution. Liquid preparations are preferred for embodiments involving sublingual or buccal administration.

In some embodiments, the pharmaceutical composition is formulated for sublingual or buccal administration. Typically, a sublingual or buccal pharmaceutical composition is a liquid; however, any other suitable dosage form known in the art may be employed including aerosols, lozenges, troches, films, foams, pastes and dissolvable tablets.

Sterile liquid form pharmaceutical compositions include sterile solutions, suspensions, emulsions, syrups and elixirs. The active ingredient(s) may be suspended in a pharmaceutically acceptable carrier, such as sterile water, sterile organic solvent or a mixture of both.

Other liquid form preparations include those prepared by combining the Cannabis extract with one or more naturally derived oils (e.g. an essential oil) or waxes. An “essential oil” is an oil derived by extraction (e.g. steam extraction, or contacting the plant material with an extractant) or pressing, which contains primarily hydrophobic, and generally fragrant, components of the plant material. Suitable naturally derived oils and waxes include Sesame oil, Olive oil, Arnica essential oil, Lavender essential oil, Lavender Spike essential oil, Frankincense essential oil, Lemongrass essential oil, Cinnamon Leaf essential oil, Rosemary Cineole essential oil, Rosemary essential oil, Bergamot essential oil, Myrrh essential oil, Sage essential oil, Coconut oil, Bees wax and Hemp oil.

The pharmaceutical compositions may be formulated for parenteral administration (e. g. by injection, for example bolus injection or continuous infusion) and may be presented in unit dose form in ampoules, pre-filled syringes, small volume infusion or in multi-dose containers optionally with an added preservative. The pharmaceutical compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems, and may contain, for example, water, ethanol, polyol (for example, glycerol, propylene glycol and liquid polyethylene glycol, and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active ingredients in the required amount in the appropriate carrier with various other ingredients such as those enumerated above, as required, followed by sterilisation. Generally, dispersions are prepared by incorporating the various sterilised active ingredients into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile suspension of the active ingredient plus any additional desired ingredients.

For oral administration, the active ingredient(s) may be incorporated with excipients and used in the form of ingestible tablets, buccal tablets, troches, capsules, elixirs, suspensions, syrups, wafers, and the like.

The amount of active ingredient(s) in a therapeutically useful pharmaceutical composition should be sufficient that a suitable dosage will be obtained. Accordingly, the active ingredient(s) are preferably provided in an effective amount.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient(s), sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient(s) may be incorporated into sustained-release preparations and formulations, including those that allow specific delivery of the active peptide to specific regions of the gut.

Aqueous solutions can be prepared by dissolving the active ingredient(s) in water and adding suitable colorants, flavours, stabilising and thickening agents, as desired. Aqueous suspensions can be made by dispersing the finely divided active ingredient(s) in water with viscous material, such as natural or synthetic gums, resins, methylcellulose, sodium carboxymethylcellulose, or other well-known suspending agents.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations for oral and/or sublingual administration. Such liquid forms include solutions, suspensions, and emulsions. These preparations may contain, in addition to the active ingredient(s), colorants, flavours, stabilisers, buffers, artificial and natural sweeteners, dispersants, thickeners, solubilising agents, and the like.

For topical administration to the epidermis the active ingredient(s) may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth (e.g. sublingual administration) include any liquid formulation described herein, preferably liquid formulations with a viscosity suitable for administration by dropper or syringe; lozenges comprising active ingredient(s) in a flavoured base, usually sucrose and acacia or tragacanth; pastilles comprising the active ingredient(s) in an inert base such as gelatin and glycerin or sucrose and acacia; and mouthwashes comprising the active ingredient(s) in a suitable liquid carrier.

For administration to the nasal cavity, solutions or suspensions may be applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension.

In the case of a spray, this may be achieved for example by means of a metering atomising spray pump. For such sprays, active ingredient(s) may be encapsulated with cyclodextrins, or formulated with other agents expected to enhance delivery and retention in the nasal mucosa.

Administration to the respiratory tract may be achieved by means of an aerosol formulation in which the active ingredient(s) are provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant. The dose of drug may be controlled by provision of a metered valve.

Alternatively, the active ingredient(s) may be provided in the form of a dry powder, for example a powder mix of the active ingredient(s) in a suitable powder base such as lactose, starch, starch derivatives such as hydroxypropylmethyl cellulose and polyvinylpyrrolidone (PVP). The pharmaceutical composition as a powder may be presented in unit dose form for example in capsules or cartridges of, e.g. gelatin, or blister packs from which the powder may be administered by means of an inhaler.

In formulations intended for administration to the respiratory tract, including intranasal formulations, the pharmaceutical composition may have a small particle size for example of the order of 5 to 10 microns or less. Such a particle size may be obtained by means known in the art, for example by micronisation.

When desired, formulations adapted to give sustained release of the active ingredient(s) may be employed.

The pharmaceutical composition may be prepared in unit dosage form. In such form, the composition is subdivided into unit doses containing appropriate quantities of the active ingredient(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Pharmaceutical compositions for parenteral administration may also be provided in unit dosage form for ease of administration and uniformity of dosage. Unit dosage form as used herein refers to physically discrete units suited as unitary dosages for the patients to be treated; each unit containing a predetermined quantity of active material calculated to produce the desired therapeutic effect in association with the required pharmaceutical excipient. The specification for the unit dosage forms are dictated by and directly dependent on (a) the unique characteristics of the active ingredient(s) and the particular therapeutic effect to be achieved, and (b) the limitations inherent in the art of compounding such an active ingredient(s) for the treatment of living patients having a diseased condition in which bodily health is impaired.

In some embodiments, the pharmaceutical composition comprises a further active ingredient. In some embodiments, the pharmaceutical composition comprises a further active ingredient other than a cannabinoid and/or terpene. Any suitable further active ingredient may be used provided that the activity of the active ingredient, THC, CBD and the terpene fraction is not diminished when combined. In some embodiments, the further active ingredient is an anti-anxiety drug or a drug suitable for treating PTSD. In some embodiments, the anti-anxiety drug is selected from a selective serotonin re-uptake inhibitor, a serotonin-norepinephrine re-uptake inhibitor, an antidepressant, a benzodiazepine, a beta-blocker, buspirone, or a monoamine oxidase inhibitor. Suitable selective serotonin re-uptake inhibitors include citalopram, escitalopram, fluoxetine, fluvoxamine, paroxetine and sertraline. Suitable serotonin-norepinephrine re-uptake inhibitors include duloxetine and venlafaxine. Suitable anti-depresants include amitriptyline, imipramine, nortriptyline, duloxetine, hydroxyzine, promethazine, carisoprodol, tripelennamine and clomipramine. Suitable Benzodiazepines include alprazolam, chlordiazepoxide, diazepam and lorazepam. Suitable beta-blockers include atenolol and propranolol. Suitable monoamine oxidase inhibitors include isocarboxazid, phenelzine, selegiline and tranylcypromine.

The practice of the present invention employs, unless otherwise indicated, conventional pharmaceutical, veterinary and medical techniques within the skill of the art. Such techniques are well known to the skilled worker and are explained fully in the literature.

### Methods of Treatment

The present invention provides a method of treating post-traumatic stress disorder (PTSD) and/or anxiety, comprising administering to a subject in need thereof an effective amount of one or more pharmaceutical compositions of the invention. In some embodiments, the method comprises treating PTSD and/or anxiety that is mediated by the endocannabinoid system, or the activity of any one of the endocannabinoid receptors, including CB1 and CB2.

Current treatments for anxiety and PTSD overlap, with antidepressants often prescribed in severe cases in conjunction with counselling or psychotherapy such as cognitive therapy, exposure therapy or eye movement desensitization and reprocessing.

In some embodiments, the therapy is given in combination with another drug suitable for treating PTSD and/or anxiety or in combination with counselling or psychotherapy such as cognitive therapy, exposure therapy or eye movement desensitization and reprocessing.

The symptoms and severity of PTSD may be diagnosed by administering a questionnaire. Subsequent completion of the same questionnaire may also be used to monitor the effectiveness of a therapy. Suitable questionnaires for PTSD and its symptoms include Primary Care PTSD Screen for DSM-5 (PC-PTSD-5), SPAN, SPRINT and the Trauma Screening Questionnaire (TSQ).

The method may comprise administering more than one pharmaceutical composition of the present invention to the patient in need thereof. For example, in some circumstances, it is preferred to administer a pharmaceutical composition high in THC and a pharmaceutical composition high in CBD to the patient. Pharmaceutical compositions high in THC (e.g. THC-rich pharmaceutical compositions) includes those that either lack any measurable content of CBD, or comprise THC in a ratio of at least about 2:1 relative to CBD, for example, about 2.5:1, 3:1, 4:1, 5:1, 6:1, 7:1, 8:1, 9:1 or 10:1 THC:CBD. Pharmaceutical compositions high in CBD (e.g. CBD-rich pharmaceutical compositions) includes those that either lack any measurable content of THC, or are those that comprise CBD in a ratio of at least about 2:1 relative to THC, for example, about 2.5:1, 3:1, 4:1, 5:1, 6:1, 7:1, 8:1, 9:1 or 10:1 CBD:THC.

In some embodiments, the method comprises administering a Cannabis extract comprising THC as primary cannabinoid and a terpene fraction and administering a Cannabis extract comprising CBD as primary cannabinoid and a terpene fraction. The terpene fraction in each of these Cannabis extracts may be the same or different in terms of amount and identity. These two Cannabis extracts may be administered simultaneously, separately or consecutively with pharmaceutical compositions of the invention. By simultaneously it is meant that each of Cannabis extracts are administered at the same time optionally in the same pharmaceutical composition. By separately it is meant that the Cannabis extract is administered at the same time in different pharmaceutical compositions and optionally by different routes of administration. By consecutively it is meant that each of pharmaceutical composition and the other active ingredient are administered separately and may be at different times. Typically, when the pharmaceutical composition and the other active ingredient are administered consecutively they are administered within 24 hours, or within 12, 8, 6, 5 or 4 hours of each other. Typically, the Cannabis extract comprising THC as primary cannabinoid and the Cannabis extract comprising CBD as primary cannabinoid are administered consecutively. The administration of the Cannabis extract comprising THC as primary cannabinoid may occur preceding a period of sleep of the subject (e.g. in the evening or at night). The administration of the Cannabis extract comprising CBD as primary cannabinoid may occur following a period of sleep of the subject (e.g. in the morning).

In some embodiments, the method further comprises a step of administering an effective amount of a pharmaceutical composition comprising THC and a terpene fraction to the patient to manage breakthrough symptoms. The pharmaceutical composition may be substantially free of CBD, or comprise THC and CBD in a ratio of THC:CBD of at least 2:1, for example, at least about 3:1, 4:1, 5:1, 6:1, 7:1, 8:1, 10:1 or higher. In some embodiments, the pharmaceutical composition for managing breakthrough symptoms a ratio of THC:CBD from about 2:1 to about 10:1 or about 3:1 to about 10:1. The pharmaceutical composition for managing breakthrough symptoms may be administered orally or via inhalation.

Also provided is an anxiolytic agent comprising THC, CBD and a terpene fraction. The anxiolytic agent may comprise any of the pharmaceutical compositions described herein in an effective amount for treating anxiety and/or PTSD in a subject in need thereof. The anxiolytic agent may therefore comprise a therapeutically effective amount of THC, CBD and the terpene fraction.

### EXAMPLE(S)

The invention will be further described by way of non-limiting example(s). It will be understood to persons skilled in the art of the invention that many modifications may be made without departing from the spirit and scope of the invention.

### Example 1

### Preparation of Oral Formulation

Oral capsules were prepared from Cannabis extracts obtained by extraction of a Cannabis plant with ethanol, followed by removal of extractant by heating in vacuo. This resulted in a solid granule, which was divided into capsules having the constitution described in Table 1.

TABLE 1
Orally bioavailable capsules

| | Amount |
|---|---|
| Ingredient | per capsule |
| THC¹ | 50 mg |
| CBD¹ | 50 mg |
| Excipient(s) | qs 150 mg |

Notes:
(¹) The THC and CBD are obtained from extraction of a Cannabis plant and each cannabinoid comprises up to about 6 wt % of a terpene fraction from the extraction process.

### Example 2

### Preparation of Oro-Mucosal Formulation

The liquid composition comprising 10 mg/mL THC and 10 mg/mL CBD was prepared from Cannabis extracts obtained by extraction of a Cannabis plant with ethanol, followed by removal of extractant by heating in vacuo. The extract was solubilized in olive oil. If required, THC and/or CBD or one or more terpenes/terpenoids may be added to the composition to provide the desired dosage.

The composition was packaged into pre-filled syringes containing the desired dosages.

### Example 3

### Preparation of Sublingual Formulation

A sublingual formulation was prepared from Cannabis extracts obtained by extraction of a Cannabis plant with ethanol. The extract was concentrated by removal of the ethanol. The resulting concentrated extract was diluted with sunflower oil to give a composition comprising 20 mg/mL THC, 2 mg/mL CBN and 1 mg/mL CBD.

### Example 4

### Effectiveness of Medicinal Cannabis Formulation on Anxiety

Twenty four (24) participants between the ages of 25 and 70 years of age with anxiety or post-traumatic stress disorder are enrolled into a double blind, randomized, placebo-controlled study. The study conducted over 9 to 12 months.

Participants are randomized into drug or placebo groups and are given doses of the composition of Example 2 in prefilled syringes to be delivered sublingually.

Amongst other endpoints, this study provides data supporting the use of cannabinoid-therapy for treating PTSD and/or anxiety. This is assessed during reviews prior to commencement of the trial and in Weeks 2 and 4 of the study. These reviews include:
Depression, Anxiety, Stress Scale (DASS)
Primary Care PTSD Screen for DSM-5 (PC-PTSD-5)
Quality of Life (Q-Les-Q)
Insomnia Severity Index (ISI)
Work & Social Adjustment Scale (WSAS)

1. A pharmaceutical composition comprising Δ⁹ tetrahydrocannabinol (THC), cannabidiol (CBD) and a terpene fraction obtained by extraction of a Cannabis plant, wherein the composition is enriched in one or the other of THC and CBD and/or one or more of the following terpenes β-caryophyllene, d-limonene, linalool and β-myrcene.

2. The pharmaceutical composition according to claim 1, wherein the ratio of THC:CBD is selected from one of the following:
a) about 10:1 to about 1:10;
b) 2:1 or more, or
c) 1:2 or more.

3. (canceled)

4. (canceled)

5. The pharmaceutical composition according to claim 1, wherein the THC is present in an amount of about 15 wt % to about 85 wt %.

6. The pharmaceutical composition according to claim 5 wherein the THC is present in an amount of about 15 wt % to about 40 wt %.

7. The pharmaceutical composition according to claim 1, wherein the CBD is present in an amount of about 15 wt % to about 60 wt %.

8. The pharmaceutical composition according to claim 7 wherein the CBD is present in an amount of about 15 wt % to about 40 wt %.

9. (canceled)

10. The pharmaceutical composition according to claim 1, wherein the total amount of THC and CBD is present is in the range of about 30 wt % to about 99 wt %.

11. The pharmaceutical composition according to claim 1 wherein the terpene fraction is present in an amount of about 0.1 wt % to about 10 wt % of the agent.

12. The pharmaceutical composition according to claim 1 wherein the terpene fraction further comprises one or more terpene selected from the group consisting of: α-pinene, p-cymene, gurjunene, a nerolidol, α-terpinene, α-phellandrene, camphene, terpinolene, 1,8-cineole, γ-terpinene, guaiol, β-ocimene, β-pinene, γ-cadinene, caryophyllene oxide and β-farnesene.

13. The pharmaceutical composition according to claim 12, wherein the terpene fraction comprises one or more terpene selected from the group consisting of: α-pinene, p-cymene, gurjunene and a nerolidol.

14. The pharmaceutical composition according to claim 1 wherein the terpene fraction further comprises α-pinene.

15. A method of treating post-traumatic stress disorder (PTSD) and/or anxiety, comprising administering to a subject in need thereof an effective amount of the pharmaceutical composition of claim 1.

16. The method of claim 15, comprising administration of a pharmaceutical composition comprising THC and a terpene fraction separately to a pharmaceutical composition comprising CBD and a terpene fraction.

17. The method according to claim 16 wherein the pharmaceutical composition comprising the THC and terpene fraction is administered within 24 hours before or after the pharmaceutical composition comprising CBD and terpene fraction.

18. The method according to claim 16 wherein the pharmaceutical composition comprising THC and a terpene fraction is administered in a period preceding sleep.

19. The method according to claim 16, wherein the pharmaceutical composition comprising CBD and a terpene fraction is administered in a period following sleep.

20. The method according to claim 16, wherein the THC and CBD is present in a ratio of THC:CBD of 2:1 or more, to manage breakthrough symptoms of PTSD and/or anxiety.

21. The method according to claim 20 wherein the pharmaceutical composition is administered orally or by inhalation.

22. A kit of parts comprising in separate parts (a) a pharmaceutical composition comprising THC and a terpene fraction, and (b) a pharmaceutical composition comprising CBD and a terpene fraction, wherein the terpene fraction comprises a terpene selected from the group consisting of β-caryophyllene, d-limonene, linalool and β-myrcene.

23. (canceled)
